(19) United States
(12) Patent Application Publication
(10) Pub. No.: US 2008/0118977 A1
(43) Pub. Date: May 22, 2008

(54) Process to cary out a cellular cardiomyoplasty

(75) Inventor: Philippe Henon, Mulhouse (FR)

Correspondence Address: DAVIS BUJOLD & Daniels, P.L.L.C., 112 PLEASANT STREET, CONCORD, NH, 03301, US

(73) Assignee: INSTITUT DE RECHERCHE EN HEMATOLOGIE ET TRANSPLANTATION, MULHOUSE (FR)

(21) Appl. No.: 11/704,107

(22) Filed: Feb. 8, 2007

Related U.S. Application Data
(63) Continuation-in-part of application No. 11/603,880, filed on Nov. 22, 2006.

(51) Int. Cl. C12N 5/02; C12N 5/08

(52) U.S. Cl. 435/372; 435/375

(57) ABSTRACT

A cellular cardiomyoplasty process based on the potential capacity of CD34⁺ cells to regenerate myocardium after acute myocardial infarct (AMI) and on their collection in blood in which the following phases are performed: Phase 1 a G-CSF-mobilization phase of CD-34+ cell is started as soon as the infarct is stabilized and its impact on heart function has been evaluated; Phase 2 a cell collecting phase is undertaken after G-CSF-mobilization; Phase 3 a cell processing phase is performed to select ex-vivo CD34+ cells and expand them in vitro to achieve around a 20-fold increase of the total number of CD34⁺ cells; Phase 4 a resuspension phase of the amplified-cell product in a final predetermined volume of autologous plasma, and Phase 5 a packaging phase of the cell suspension in a sterile syringue for reinjection to the patient.

### FIELD OF THE INVENTION

The present invention concerns a cellular cardiomyoplasty process based on the potential capacity of CD34⁺ cells to regenerate myocardium after acute myocardial infarct (AMI) and on their collection in blood.

### BACKGROUND OF THE INVENTION

Many spontaneous or injury-related diseases are due to particular types of cells not functioning correctly. They currently have slightly or non-efficient treatment options, and millions of people worldwide are desperately waiting to be cured. The new concept of “regenerative medicine”, which proposes to use stem cells for regeneration of damaged tissues or organs, could treat a patient in such a way that both the immediate problem is corrected and the normal physiological processes are restored without the need of subsequent drug or similar treatment.

Embryonic stem cells, theoretically capable of producing any type of more mature cells, tissues and organs, should of course be the best candidates for regenerative medicine. However, numerous unresolved ethical and technical problems make their therapeutic use within the forthcoming years illusive.

On the contrary, a long standing biological dogma—that a cell, once committed, cannot alter its fate—has been recently challenged: a host of recent experimental papers have indeed suggested that stem cells from various adult tissues could be reprogrammed and eventually match the versatility of those derived from embryos. Among those “adult” stem cells (ASCs), hematopoietic stem cells (HSCs) are the only ones to have been presently isolated in animals and in humans. They normally reside in the bone marrow but, under some conditions, can migrate to other tissues through blood flow. Recent experimental data suggest that, under certain conditions of organic stress, they might dedifferentiate or transdifferentiate to tissues other than hematopoietic bone marrow.

Chronic heart failure (CHF), most often related to a large size AMI is undoubtedly the most important health problem in developed countries. Its prevalence can effectively reach up to 2% of the total population in European countries, with a dramatic increase in elderly people. About 5 million new patients are diagnosed with AMI each year in the United States, of whom around 10% present a large infarct associated with rapid development of subsequent CHF. Under such conditions, their morbidity rate is very high, leading to an annual related-cost of more than 25 billion USD. And despite recent and significant therapeutical progresses, no medication or surgical procedure can restore viability, vascularization and functional contractility of the myocardial necrotic lesion. About 35% of these patients will die within one year post AMI, and 50% within 4 years post AMI; up to 200,000 US patients annually die from this condition. Moreover, even though the number of patients rapidly necessitating heart transplant is duly increasing, a decreasing number may effectively benefit from this procedure due to lack of donors. For example, 500 patients on average are waiting for heart transplantation every year in France, and 4,000 patients in USA. Also, a study recently realized by the American Heart Association has evaluated the number of cardiac patients who would require either extra-corporal (artificial heart devices) or intra-corporal (implantable defibrillators) heart-assistance systems to be around 100,000 in USA, which would represent a 3 billion dollar cost.

Most of these patients, and more particularly those with post-AMI CHF might duly benefit from cellular cardiomyoplasty.

### PRIOR ART

The clinical use of embryonic stem cells for restoration of cardiac function after AMI is presently impossible as mentioned before.

In contrast, a growing number of studies have recently provided experimental data strongly suggesting that HSCs would be capable of transdifferentiation (“cell plasticity”). Jackson et al., (Jackson K. A. et al. Regeneration of ischemic cardiac muscle and vascular endothelium by adult stem cells. J. Clin. Invest. 2001: 107:1394-5-1402) have shown that progeny of murine HSCs transplanted in mice, previously lethally irradiated and rendered ischemic by transient coronary occlusion, migrated and differentiated to cardiomyocytes and endothelial cells into ischemic cardiac muscle and blood vessels.

Lagasse et al., (Lagasse E. et al. Purified hematopoietic stem cells can differentiate into hepatocytes in vivo. Nat Med 2000; 6:1229-1234) have injected “purified” murine Bone Marrow (BM) c-kit^{hi} Thy^{lo} Lin⁻ Sca-1⁺ cells in the blood stream of mice with fatal hereditary tyrosinemia. As few as 50 of these cells not only led to the restoration of the hematopoietic system but, more surprisingly, also seemed to cure the tyrosinemia-related liver disease.

Kocher et al., (Kocher et al. Neovascularization of ischemic myocardium by human bone marrow-derived angioblasts prevents cardiomyocyte apoptosis, reduces remodelling and improves cardiac function. Nat Med 2001; 7:430-433) have purified CD34⁺ Lin⁻ cells from human bone marrow and further injected these cells in the blood stream of NOD/Scid mice 48 h after they had undergone an experimental infarction: a part of these cells (or their progeny) seemed to transdifferentiate to endothelial cells contributing to a further neo-angiogenesis and myocardial revascularization accompanied by a significant improvement of the cardiac function.

In a study which would have appeared as maybe the most significant, Orlic et al., (Orlic D et al. Bone marrow cells regenerate infarcted myocardium. Nature 2001; 410:701-704) have reported that hematopoietic stem cells (CD34⁺ Lin⁻ c-kit^{hi}), purified from transgenic male mice bone marrow, further injected directly into female mice experimentally-damaged myocardium, gave rise to cells exhibiting markers and morphology of immature cardiomyocytes, endothelial cells and smooth muscle cells (Orlic D et al. Bone marrow cells regenerate infarcted myocardium. Nature 2001; 410:701-704). The injected hearts showed a 35% improvement of their function, with appearance of neo-angiogenesis into the injured myocardial zone.

Injecting bone marrow stem cells into an injured heart thus would potentially represent a new therapy. This experimental study has triggered the launch of numerous clinical ones to investigate the effect of directly injecting these cells into the damaged heart muscle of patients following a heart attack. However, two recent studies in mice (Murry G. E. et al. Haematopoietic stem cells do not transdifferentiate into cardiac myocytes in myocardial infarcts. Nature 2004; 428:664-668 and Balsam L. B. et al. Haematopoietic stem cells adopt mature haematopoietic fates in ischemic myocardium. Nature 2004; 428:668-673) and two commentaries (Chien K R. Stem cells: lost in translation. Nature 2004; 428:607-608, Unlisted authors. No consensus on stem cells. Nature 2004; 428-587) have challenged the ability of bone marrow cells to differentiate into myocytes and coronary vessels suggested by Orlic et al, and claimed that their original findings were a collection of artifacts. The claim has also been made that bone marrow cells might acquire a cell phenotype different from the blood lineages only by fusing with resident cells (Terada N. et al. Bone marrow cell adopt the phenotype of other cells by spontaneous cell fusion. Nature 2002; 416:542-545, and Ying Q. L. et al. Changing potency by spontaneous fusion. Nature 2002; 416:545-548). These reports might raise serious concerns regarding the feasibility of using stem cells derived from the bone marrow to drive cardiac regeneration. Balsam et al even concluded extremely severely their report by claiming “without additional pre-clinical experimental data, all clinical trials are premature, with emphasis, and may in fact place a group of sick patients at risk”! (Balsam L. B. et al. Haematopoietic stem cells adopt mature haematopoietic fates in ischemic myocardium. Nature 2004; 428:668-673).

Nevertheless, Kajstura et al, from the same group as Orlic, have countered this attack in a paper published in early 2005 (Kajstura J. et al. Bone Marrow cells differentiate in cardiac cell lineages after infarction independently of cell fusion. Circ Res 2005; 96:127-137). They used c-kit+ bone marrow cells obtained from male transgenic mice and transplanted them in recipient female infarcted hearts. Using GFP and the Y-chromosome as markers of the progeny of c-kit+ cells, this group demonstrated that the transplanted cells efficiently differentiate, independently from cell fusion, into as much as 4.5 million biochemically and morphologically differentiated myocytes, together with coronary areterioles.

Thus, the vigorous debate about bone marrow stem cells transdifferentiation is far from being closed, and is not convincingly underpinned by the current ongoing clinical studies.

From 2002, an increasing number of clinical studies using BM mononuclear cells (MNC) have been launched to investigate the effect of injecting these cells either directly into the damaged heart or in the infarct-related artery in patients following a heart attack. Most were non-randomized pilot studies (Strauer B. E. et al. Repair of infarcted myocardium by autologous intracoronary mononuclear bone marrow cell transplantation in humans. Circulation 2002; 106:1913-1918, and Perin E. C. et al. Improved exercise capacity and ischemia 6 and 12 months after transendocardial injection of autologous bone marrow mononuclear cells for ischemic cardiomyopathy. Circulation 2004; 110(suppl 1):II213-II218), a majority of studies have been now completed while several are still ongoing. All indicated feasibility, safety and, with the exception of one (Kuethe F. et al. Lack of regeneration of myocardium by autologous intra-coronary mononuclear bone marrow cell transplantation in humans with large anterior myocardial infarctions. Int J Cardial 2004; 97:123-127), enhanced cardiac functional recovery, although at various degrees.

A few randomized studies have been more recently reported, also with contradictory results (Schachinger V. et al. Transplantation of progenitor cells and regeneration enhancement in acute myocardial infarction. Final one-year results of the TOPCARE-AMI trial. J Am Coll Cardiol 2004; 44:1690-1699 and Janssens S. et al. Autologous bone marrow derived stem cell transfer in patients with ST-segment elevation myocardial infarction: double blind, randomized controlled trial. Lancet 2006; 367:113-121). For example, results of a randomized open-label study indicated improvement of LV systolic function but not of LV remodeling after transfer of BM-derived stem cells (Wollert K. C. et al. Intracoronary autologous bone marrow cell transfer after myocardial infarction: the BOOST randomized controlled clinical trial. Lancet 2004; 364:141-148). Moreover, the control group usually did not reproduce the exact conditions of the group to which cells were transferred (Schachinger V. et at. Transplantation of progenitor cells and regeneration enhancement in acute myocardial infarction. Final one-year results of the TOPCARE-AMI trial. J Am Coll Cardiol 2004; 44:1690-1699 and Wollert K. C. et al. Intracoronary autologous bone marrow cell transfer after myocardial infarction: the BOOST randomized controlled clinical trial. Lancet 2004; 364:141-148). The only study fully reproducing those, including bone marrow aspiration and a placebo intra-coronary injection was very recently published (Janssens S. et al. Autologous bone marrow derived stem cell transfer in patients with ST-segment elevation myocardial infarction: double blind, randomized controlled trial. Lancet 2006; 367:113-121). This meticulously performed double-blind placebo-controlled study, in which autologous BM-derived cell infusion was done in the peri-infarct period, provides confirmation of the feasibility and safety of the technique; additionally, there appeared to be no increase in ischemia, infarction, or arrhythmya. BMSC transfer was associated with a significant reduction in myocardial infarct size and a better recovery of regional systolic function. However, there was no difference in myocardial perfusion and metabolism increase between both groups globally studied. But Janssens et al enrolled in this study a low-risk population in which 38% had infarcts in the right coronary artery and the average LVEF was about 55%. Thus, ventricular function in these patients was probably too well preserved to expect significant functional improvement from BMSC infusion. Janssens et al. have moreover given themselves additional data to support this concept: metabolic activity was indeed increased in the treated patients, compared with control patients, when the analysis was limited to the largest nine infarcts in each group. Similarly, there was an increased likelihood of improvement in wall-motion index in treated patients compared with controls, when the segment had more than 75% transmural involvement.

In fact, patients enrolled to date in most reported studies have been at relatively low risk for death or development of congestive heart failure, when it would have been more prudent and probably more significant to exclusively enroll patients at high risk (Penn M. S. Stem cell therapy after acute myocardial infarction: the focus should be on those at risk. Lancet 2006; 367:27-88).

And finally, accurate evaluation of the role potentially played by reinjected cells in cardiac function improvement is unlikely to rise from these pilot or randomized studies for various reasons:
It is difficult to demonstrate myocardial regeneration in humans in the absence of cardiac biopsy and/or ethically-approved biological markers,
As the infarction area was reperfused in all studies, either by bypass surgery or by repermeabilization of the infarct-related artery, it is impossible to determine if the potential neo-vascularization generally observed was related to the reperfusion or actually to a cell-related neo-angiogenesis mechanism,
BM-MNCs harvests represent in fact a cellular “soup” containing different types of ASC: “true” HSCs, mesenchymal stem cells, other stroma cells, and maybe more. It is thus impossible to determine which cell type would actually be implied in potential myocardial regeneration and revascularization,
Also, questions have arisen about whether the improvement in ejection fraction observed in most studies was due to the procedure used to deliver BM-MNCs or the BM-MNCs themselves. Cell reinfusion techniques could indeed induce further expression of stem-cell homing signals within the myocardium, resulting in transient healing response.

Regarding remarks made above, it would be preferable to use selected stem cells rather than to reinfuse a “melting pot” of various stem cells. It would indeed allow a better determination which type(s) of cells—if any—is actually involved in potential cardiac improvements.

Moreover, collecting blood CD34+ cells after mobilization rather than BM CD34+ cells has several advantages:
leukapheresis products contain much more CD34+ cells and consequently their positive selection is easier and much more productive,
it is much less painful for the patient and avoids the need for anesthesia.

Since 2003, several papers have strongly suggested that human CD34+ cells might transdifferentiate either into endothelial cells or into cardiomyocytes. Cocultivating human blood-derived endothelial progenitor cells or CD34+ cells with rat cardiomyocytes, Badorff et al., (Badorff C. et al. Transdifferentiation of blood-derived human adult endothelial progenitor cells into functionally active cardiomyocytes. Circulation 2003; 107:1024-1032) have shown that these cells can transdifferentiate in vitro into functionally active cardiomyocytes, identified by their expression of α-sarcomeric actinin and cardiac troponin I. This transdifferentiation was mediated by cell-to-cell contact, but not by cellular fusion. Pesce et al., (Pesce et al. Myoendothelial differentiation of human umbilical cord blood-derived stem cells in ischemic limb tissues. Circ Res 2003; 93: e51-e62) have demonstrated that freshly isolated human cord blood CD34+ cells injected into ischemic adductor muscles gave rise to endothelial and, unexpectedly, to skeletal muscle in mice: the treated limbs exhibited enhanced arteriole length density and regenerating muscle fiber density. More importantly in view of what we will propose later, endothelial and myogenic differentiation ability was maintained in CD34+ cells after ex vivo expansion. Yeh et al., have, in a first study, investigated whether adult human PB CD34+ cells could transdifferentiate into human cardiomyocytes, mature endothelial cells and smooth muscle cells in vivo (Yeh E. T. et al. Transdifferentiation of human peripheral blood CD34+-enriched cell population into cardiomyocytes, endothelial cells, and smooth muscle cells in vivo. Circulation 2003, 108:2070-2073). They have first created myocardial infarction in SCID mice by occluding the left anterior descending coronary artery, and then they have injected human adult PB CD34+ cells into the tail vein. Two months after injection, cardiomyocytes, and endothelial cells bearing human leucocyte antigen were identified in the infarct and peri-infarct regions of the mouse hearts. In a separate experiment, CD34+ cells were injected intraventricularly into mice without experimental myocardial infarction: HLA-positive myocytes and smooth muscle cells could only be identified in one of these killed mice. Thus, transdifferentiation would likely be dependent on local tissue injury.

However, in another paper, the same group was backed up a little regarding their previous conclusions (Zhang S. et al. Both cell fusion and transdifferentiation account for the transformation of human peripheral blood CD34-positive cells into cardiomyocytes in vivo. Circulation 2004; 110:3803-3807). They effectively observed in the same experimental conditions that 73% of nuclei derived from HLA⁺ and Troponin+ or Nkx-2.5⁺ cardiomyocytes, contain both human and mouse X-chromosomes and 24% only contain human X-chromosome. In contrast, the nuclei of HLA⁻, Troponin T⁺ cells only contain mouse X-chromosome. Furthermore, 94% of endothelial cells derived from CD34⁺ cells only contain human X-chromosome. Thus, the authors now concluded that both cell fusion and transdifferentiation might account for the transformation of peripheral blood CD34⁺ cells into cardiomyocytes in vivo.

Of course, these conclusions do not really clarify the debate on stem cell plasticity.

Another option would be that immature endothelial and myocytic progenitors could already exist in the bone marrow. In case of any organic stress, they could be mobilized into circulating blood and would home to the injured organ, for example to the myocardial infarcted region.

Asahara et al, have been the first ones to show in 1997 the existence in circulating blood of healthy human volunteers of MNCs, which can acquire in vitro an endothelial cell-like phenotype and can be incorporated in vivo into capillaries (Asahara T. et al. Isolation of putative progenitor endothelial cells for angiogenesis. Science 1997; 275:964-967). These cells expressed both CD34 and vascular-endothelial growth factor (VEGFR-2), which are shared by embryonic endothelial progenitors and HSCs. He has then postulated that these CD34⁺/VEGFR-2+ cells might be early endothelial progenitor cells (EPCs), although Flamme et al. had already shown—but in animal experimental conditions—that both CD34 and VEGFR-2 were also expressed on mature endothelial cells (Asahara T. et al. Isolation of putative progenitor endothelial cells for angiogenesis. Science 1997; 275:964-967). More recently, Peichev et al demonstrated in an outstanding study that an average of 2% mobilized PB-CD34⁺ cells were VEGFR-2⁺ and that most of these cells also express the hematopoietic stem cell marker AC133, which is present on immature hematopoietic cells too, but absent on mature endothelial or differentiated hematopoietic cells (Peichev M. et al. Expression of VEGFR-2 and AC133 by circulating human CD34+ cells identifies a population of functional endothelial procursors. Blood 2000; 95:952-958). Thus, coexpression of VEGFR-2 and AC133 on CD34⁺ cells phenotypically identifies a unique population of EPCs. In addition, virtually all the CD34⁺/VEGFR-2 cells express the chemokine receptor CXCR₄ and migrate in response to stromal-derived factor-1 (SDF-1) or VEGF. Using an in vivo human model, Peichev et al have found as well that the neo-intima formed on the surface of left-ventricular assist devices was colonized with AC133⁺/VEGFR-2⁺ cells. Thus, all these data strongly suggest that circulating CD34⁺ cells expressing VEGFR-2 and AC133 constitute a phenotypically and functionally distinct population of circulating endothelial progenitor cells that might contribute to neo-angiogenesis (angioblast-like cells).

Going along the same line, the inventor of the present invention has confirmed the presence of CD34⁺ cells expressing both VEGFR-2 and AC133 (average 0.6%, range: 0.21-1.16) in leukapheresis products (LKP) yielded after G-CSF mobilization in cancer patients (See Table 1 hereunder and FIG. 1).

TABLE 1
Quantification of total CD34⁺ cells and CD133⁺ and CD133⁺/VEGFR-2⁺ subsets in “purified” or “not purified” LKP from patients
with cancer after chemotherapy + G-CSF mobilization

| | | | Controls | | | | | | | |
|---|---|---|---|---|---|---|---|---|---|---|
| | | | CD34+ selection | | | | | Total LKP | | Average |
| Type of cells | Evaluation parameters | | 1 | 2 | 3 | 4 | 5 | 6 | 7 | value |
| Total CD34⁺ | Selection Purity | (%) | 98.4 | 87.3 | 90.3 | 80.5 | 97.9 | — | — | 90.9 |
| cells | Viability | (%) | 100 | 97 | 98 | 90 | 99 | 99 | 100 | 97.6 |
| | Nb of total CD34⁺ cells | (×10⁶) | 359.8 | 495.1 | 102 | 225 | 340.7 | 182 | 11.9 | 254.2 |
| CD34⁺ | CD133⁺ | % | 90.6 | 90.5 | 84.2 | 94.2 | 85.8 | 78.9 | 77.8 | 86 |
| subsets | | Absolute nb (×10⁶) | 331 | 513.2 | 85.1 | 212 | 292.7 | 143.6 | 9.2 | 226.7 |
| | CD133⁺/VEGFR-2⁺ | % | 0.21 | 0.20 | 0.16 | 1.03 | 0.23 | 1.16 | 1.15 | 0.59 |
| | | Absolute nb (×10⁶) | 0.787 | 1.13 | 0.18 | 2.27 | 0.78 | 3.39 | 0.136 | 1.24 |

The eventuality that other PB-CD34⁺ cell subsets might also co-express myocytic and/or cardiomyocytic markers had not been suggested so far and thus remained hypothetic. For example, when they investigated whether cord blood- or PB-CD34⁺ cells could transdifferentiate into cardiomyocytes (see above), neither Pesce (Pesce et al. Myoendothelial differentiation of human umbilical cord blood-derived stem cells in ischemic limb tissues. Circ Res 2003; 93: e51-e62) nor Yeh (Yeh E. T. et al. Transdifferentiation of human peripheral blood CD34+-enriched cell population into cardiomyocytes, endothelial cells, and smooth muscle cells in vivo. Circulation 2003, 108:2070-2073) have taken the precaution to verify if already differentiated cardiomyocyte progenitors might in fact have already pre-existed within the total CD34⁺ cells they reinfused; this eventuality would have moreover harmed their transdifferentiation hypothesis.

Meticulously screening the same LKP products that were used for EPCs evaluation, recently it was shown that minor fractions of mobilized CD34⁺ cells co-expressed either Desmin (muscular marker) with an average of 0.39% cells (range 0.01-1.16%) or Troponin-T (cardiomyocyte marker) (0.17 and 0.69% respectively in 2 LKPs). (See Table 2 hereunder and FIGS. 2A and 2B).

TABLE 2
Quantification of total CD34⁺Desmin⁺ and CD34⁺Troponin-T⁺ subsets in “purified” or “not purified” LKP from patients with cancer after
chemotherapy + G-CSF mobilization

| | | | Controls | | | | | | | | | |
|---|---|---|---|---|---|---|---|---|---|---|---|---|
| | | | CD34+ selection | | | | | | Total LKP | | | Average |
| Type of cells | Evaluation parameters | | 1 | 2 | 3 | 4 | 5 | 6 | 7 | 8 | 9 | value |
| Total CD34⁺ | Selection Purity | (%) | 98.4 | 87.3 | 90.3 | 80.5 | 97.9 | — | — | — | — | 90.9 |
| cells | Viability | (%) | 100 | 97 | 98 | 90 | 99 | 99 | 100 | 97 | 98 | 97.6 |
| | Nb of total | (×10⁶) | 359.8 | 495.1 | 102 | 225 | 340.7 | 182 | 11.9 | 181.3 | 362.6 | 251.2 |
| | CD34⁺ cells | | | | | | | | | | | |
| CD34⁺ | Desmin⁺ | % | 0.03 | 0.04 | 0.01 | 0.05 | 0.01 | 1.16 | 1.15 | 0.68 | 0.36 | 0.89 |
| subsets | | Absolute nb (×10⁶) | 0.11 | 0.23 | 0.01 | 0.12 | 0.03 | 3.39 | 0.14 | 1.23 | 1.1 | 0.77 |
| | Troponin-T⁺ | % | ND | ND | ND | ND | ND | ND | ND | 0.69 | 0.17 | — |
| | | Absolute nb (×10⁶) | ND | ND | ND | ND | ND | ND | ND | 1.3 | 0.62 | — |

However, as the intracytoplasmic expression of these 2 markers makes impossible the appliance of double marking flow-cytometry, it was not possible to determine if they are both co-expressed by the same CD34⁺ cells.

Furthermore, applying RT-PCR on the same LKP, messenger RNA either for eNOS and KDR (endothelial genes) or Nkx2-5 and Troponin-T (cardiomyocyte genes) were detected every time, thus confirming the mobilization in blood of early differentiated cardiomyocytic progenitors (Table 3).

TABLE 3
RT-PCR detection of endothelial (KDR and eNOS) and cardiac
(Troponin T and Nkx-2.5) cell subsets

| | Genes | | | |
|---|---|---|---|---|
| Patients | KDR | eNOS | Nkx2-5 | cTnT |
| Controls | 1.0 | 1.0 | 1.0 | 1.0 |
| (100% positive cells) | | | | |
| 1 | 2.7 · 10⁻⁴ | 2.8 · 10⁻³ | 2.0 · 10⁻⁴ | ND |
| 2 | 6.9 · 10⁻⁵ | 1.4 · 10⁻³ | 5.4 · 10⁻⁵ | 3.9 · 10⁻⁷ |
| 3 | 2.1 · 10⁻⁵ | 9.4 · 10⁻⁴ | 4.9 · 10⁻⁴ | ND |
| 4 | 6.5 · 10⁻⁴ | 1.5 · 10⁻³ | 3.7 · 10⁻⁶ | 4.5 · 10⁻⁷ |
| 5 | 1.8 · 10⁻⁵ | 7.2 · 10⁻⁴ | 5.3 · 10⁻⁵ | 2.0 · 10⁻⁷ |
| 6 | ND | 3.6 · 10⁻³ | 1.0 · 10⁻⁵ | 3.7 · 10⁻⁷ |

Thus, according to all these data, it is now possible to reasonably conclude that total PB-CD34⁺ cells mobilized in blood by G-CSF, also contain, beside a majority of “true” HSCs, minor subsets recognized either as endothelial progenitor cells and mature endothelial cells, or myocytic/cardiomyocytic progenitor cells. Both these subsets might of course play an important role for further myocardic regeneration.

Cellular cardiomyoplasty clinical assays using peripheral blood stem cells instead of BM mononuclear cells are fewer.

The inventor of the present invention is the first to have experimented this different approach and the preliminary data were first presented during the annual meeting of the International Society for Experimental Hematology in July 2003 (Hénon Ph. Mobilized and purified autologous blood CD34+ cell transplantation for myocardial regeneration. Personal presentation at the 32nd Annual Meeting of the International Society for Experimental Hematology, Paris, France, Jul. 5-8, 2003) and the annual meeting of the American Society of Hematology in December 2003 (Hénon Ph. et al. Intracardiac reinjection of purified autologous blood CD34+ cells mobilized by G-CSF can significantly improve myocardial function in cardiac patients. Blood 2003; 102:11, 1208a).

Two other groups have further developed a similar approach, also using PBSC mobilized by G-CSF, but with variants: the group of Pompilio et al has proposed to positively select the CD133 subpopulation to exploit its high potential for multiplication and angiogenic differentiation as Stamm has done from BM cells (Stamm C. et al. Autologous bone marrow stem cell transplantation for myocardial regeneration. Lancet 2003; 361:45-46). They did not observe any adverse effect due to cytokine administration nor to apheresis procedure. However, if they observed an improvement in reperfusion, they did not obtain any significant improvement of left ventricular contractility (Pompillo G. et al. Autologous peripheral blood stem cells transplantation for myocardial regeneration: a novel strategy for cell collections and surgical injection. Ann Thorac Surgery 2004; 78:1812-1813). Kang et al have prospectively randomized into 3 groups 27 patients with myocardial infarction who underwent coronary stenting: one undergoing intra-coronary reinfusion of PB cells mobilized by G-CSF, the 2^{nd} was administered G-CSF alone, the 3^{rd} was a control group (undergoing only stenting) (Kank et al. Effects of intra-coronary infusion of peripheral blood stem cells mobilized with granulocyte-colony stimulating factor on left ventricular systolic function and restenosis after coronary stenting in myocardial infarction: the MAGIC cell randomized clinical trial. Lancet 2004; 363:751-756). Exercise capacity, myocardial perfusion and systolic function improved significantly in patients who received cell infusion. However, an unexpectedly high rate of in-stent restenosis at culprit lesion occurred in patients who received G-CSF (Groups 1 and 2), related to a neo-intima hyperplasia. This late adverse effect could be due to the combination of reinjection of non-selected blood cell products, containing many neutrophils, monocytes and platelets, of intra-coronary stenting, and of possible acceleration of neo-intima growth with bare metal stents by G-CSF administration. Such a combination should probably be avoided, but likely does not challenge the administration of G-CSF only for mobilization without further stenting.

The present invention attempts to overcome the disadvantages of the prior art and to offer a solution to simply, efficiently, reliably and at moderate cost allow treatment of chronic cardiac failure by carrying out an innovative cellular cardiomyoplasty process.

### EXPLANATION OF THE INVENTION

The cellular cardiomyoplasty process based on the potential capacity of CD34⁺ cells to regenerate myocardium after acute myocardial infarct (AMI) and on their collection in blood of the invention is characterized in which the following phases are performed:

Phase 1 a phase of CD-34+ cell mobilization by G-CSF is started as soon as the infarct is stabilized and its impact on heart function has been evaluated,

Phase 2 a cells collecting phase is undertaken after the G-CSF mobilization,

Phase 3 a cells processing phase is performed to select ex-vivo CD34+ cells and expand them in vitro to achieve around a 20-fold increase of the total number of CD34⁺ cells,

Phase 4 a resuspension phase of the amplified cell product in a final predetermined volume of autologous plasma, and

Phase 5 a packaging phase of the cell suspension in a sterile syringe for reinjection to the patient.

According to a preferred manner to utilize the process of the above invention, the G-CSF administration is performed at least between 3-5 days after AMI.

The cells collection phase is preferably undertaken at least on the 6^{th} day of G-CSF mobilization.

The cells collection phase is preferably performed by withdrawing total blood at a final total volume of at least 200 ml.

The cells collection phase is preferably performed by several sequential venous punctures within a term of about 12 hours.

The cells collection phase is preferably performed by at least 3 to 4 sequential venous punctures.

The in vivo expansion of the CD 34+ cells of phase 3 is preferably performed during a two weeks period.

The resuspension phase is preferably performed in a final predetermined volume of between 5 and 15 ml and preferably 10 ml of autologous plasma.

The reinjection of phase 5 is preferably performed within between 5 and 18 and advantageously about 12 hours following packaging.

### BRIEF DESCRIPTION OF THE DRAWINGS

The present invention and its advantages will be more apparent from the following description of the preferred embodiment, with reference to the attached drawings, provided by way of non-limiting examples, wherein:

FIG. 1 represents a diagram of flowcytometry: expression of VEGFR-2(KDR) and CD133 by circulating human CD34+ cells,

FIG. 2A represents a diagram of flowcytometry: expression of Desmin by circulating human CD34+ cells,

FIG. 2B represents a diagram of flowcytometry: expression of Troponin T by circulating human CD34+ cells,

FIG. 3 represents schematically the clinical Phase I protocol,

FIG. 4 represents patient N° 2 PETScan,

FIG. 5 represent a diagram illustrating the process of the invention, and

FIG. 6 represents an ex vivo expansion of CD34+ PBSC populations.

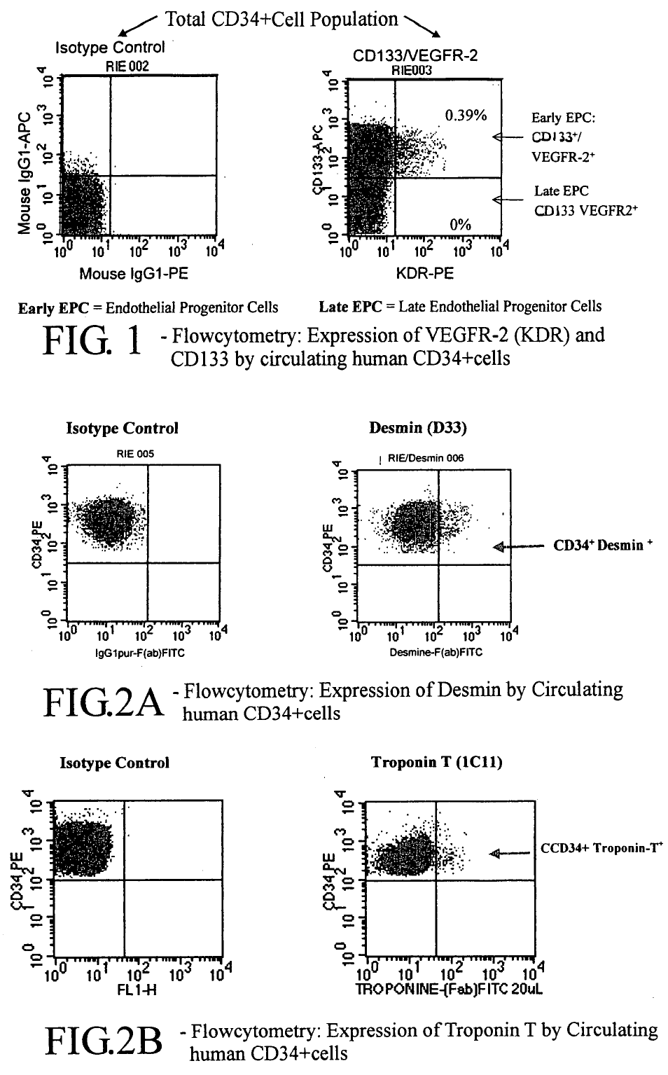

### ILLUSTRATIONS OF THE INVENTION

Intending to clinically develop the preliminary data, a clinical phase-I trial to assess the feasibility, the safety, and the potential impact on cardiac function of G-CSF-mobilization, collection, selection and intra-cardiac reinjection of autologous blood CD34⁺ cells was started.

Ten patients were scheduled according to following criteria: transmural AMI greater than 2 weeks; isotopic left-ventricular ejection fraction (LVEF) ≦35%; distinct area of akinesis corresponding to the area of infarction in the left ventricular wall; candidates for coronary artery by-pass graft (CABG); age <70 years; class IV exercise-capacity according to the New York Heart Association (NYHA) criteria. Patients were assessed before entering the trial and at 6 months post-surgery with left-heart catheterisation, three-dimensional echocardiography, ²⁰¹thallium scintigraphy, and Positron Emission Tomoscanography (PETScan) after successive intravenous injections of ¹⁸FI-FDG and of ²⁰¹Ti-Chloride to evaluate both myocardial viability and perfusion.

After patient's informed consent, mobilization of CD34⁺ cells was started 7 days before the CABG by sub-cutaneous injections of G-CSF (Granocyte® kindly provided by Chugai France), 5 μg twice daily for 5 consecutive days. Early morning of the 6^{th} day, a blood sample was withdrawn for flow-cytometry (FCM) CD34-monitoring. The apheresis, performed with a Fresenius AS104 Cell Separator, was began as soon as monitoring results were provided, with the goal to collect at least 100×10⁶ cells recommended for a further satisfactory cell selection procedure. The content of the apheresis product was immediately evaluated by FCM to ensure the expected collection of CD34⁺ was achieved. When it was not, a 2^{nd} apheresis session was performed early in the morning of the 7^{th} day. In any case, the bag containing the 6^{th} day-cell product was stored at 4° C. until the CD34⁺ selection. Patients remained hospitalized all along the mobilization/collection period in intensive cardiological care unit so as to immediately correct any unexpected side effect, which might occur with these particular category patients.

Regarding CD34 selection, the whole apheresis product was incubated with an anti-CD34 monoclonal antibody (MoAb) conjugated with ferrite beads and passed through the clinical Isolex 300i magnetic cell-separation device (Baxter-France). Then CD34⁺ cells were released from beads and resuspended in autologous plasma at a final graft volume of 15-20 ml. An additional 5 ml sample was used for CMF quantification of cells recognized when labeled with AC133 and VEGFR-2 (KDR) MoAbs to have a high angiogenetic potential, or of cells labeled with D33 MoAb, which react, with Desmin in striated muscle cells and those labeled with 1C11 MoAb which reacts with Troponin-T (Table 4).

TABLE 4
CMF determination of endothelial and muscle progenitors in the CD34+ fraction
reinfused in 5 patients after AMI

| | | CD34+ | | | | | | | |
|---|---|---|---|---|---|---|---|---|---|
| | | | | CD133+/ | | | | | |
| | CD34+ | CD133+ | | VEGFR+ | | Desmine+ | | Troponine+ | |
| Patients | (×10⁶) | % | *10⁶ | % | *10⁶ | % | *10⁶ | % | *10⁶ |
| WEN. Fe. | 29.10 | 61.90 | 18.00 | 0.02 | 0.006 | 0.10 | 0.03 | 0.54 | 0.16 |
| RIE. M-R | 40.30 | 87.70 | 35.30 | 0.39 | 0.16 | 0.11 | 0.04 | ND | ND |
| KHE. AI. | 43.80 | 83.30 | 36.50 | 0.28 | 0.12 | 0.06 | 0.03 | 0.51 | 0.22 |
| RIN. Fr. | 107.60 | 63.59 | 68.32 | 0.09 | 0.10 | 1.18 | 1.27 | 0.19 | 0.20 |
| MAL. M. | 41.00 | 44.75 | 18.34 | 1.12 | 0.46 | 0.26 | 0.11 | 0.54 | 0.22 |

KDR, eNOS, Troponin-T and Nkx 2.5 RNA-messenger were also evaluated in parallel using molecular biology methods:

TABLE 5
RT.PCR detection of endothelial (KDR and eNOS) and cardiac
(Nkx-2-5 and Troponin T) cell subsets in the CD34⁺ cell
fraction reinfused in 5 patients after AMI.

| | KDR | eNOS | Nkx2-5 | Troponin-T |
|---|---|---|---|---|
| WEN. Fe. | 2.7 · 10⁻⁴ | 2.8 · 10⁻³ | 1.8 · 10⁻⁴ | ND |
| RIE. M-R. | 6.9 · 10⁻⁵ | 1.4 · 10⁻³ | 5.4 · 10⁻⁵ | 3.9 · 10⁻⁷ |
| KHE. Ai. | 2.1 · 10⁻⁵ | 9.4 · 10⁻⁴ | 4.9 · 10⁻⁴ | ND |
| | 5.1 · 10⁻⁵ | 4.3 · 10⁻⁴ | 7.1 · 10⁻⁴ | ND |
| RIN.Fr. | 6.5 · 10⁻⁴ | 1.5 · 10⁻³ | ND | 4.5 · 10⁻⁷ |
| MAL.Fr. | 4.2 · 10⁻⁴ | 4.2 · 10⁻³ | ND | ND |

ND: Non detected
Data provided by both techniques confirmed the presence in patients graft products of endothelial as well as cardiomyocyte progenitors, as predetermined in LKP controls.

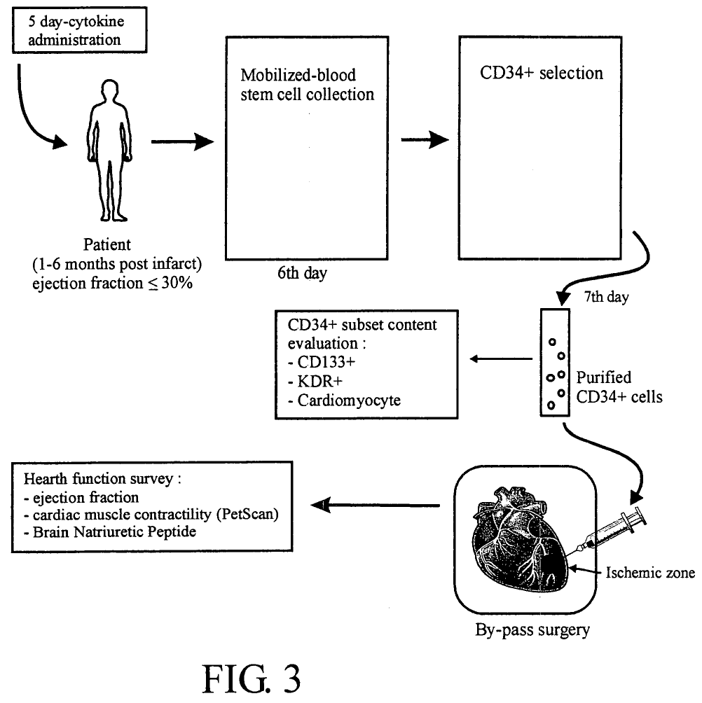

The clinical protocol is summarized on FIG. 3 which schematically represents, by way of an example, a preferred protocol of the process of the invention that relates to a process of repairing ischemic heart with mobilized purified CD34+ cells.

According to the FIG. 3, the first step concerns the administration of 5 day-cytokine. The second step concerns a mobilized-blood stem collection performed at the 6^{th} day. The third step concerns the CD34+ selection. The fourth step concerns the obtention of purified CD34+ cells at the 7^{th} day. The fifth step concerns the CD34+ subset content evaluation: CD133+; KDR+; Cardiomyocyte and the injection in the Ischemic zone of the patient which by-passes surgery.

Once the study had been approved, five consecutive patients have been enrolled in the study. The first 3 patients benefit now from a significant 3-year follow-up.

Their data are detailed on Table 6. The first patient was enrolled for a compassionate reason, owing to his relative youth (39 y-old), but as he had undergone AMI 8 years ago, he was rather considered as a negative control. The second patient was the most severely affected, with a tri-troncular occlusion, which had occurred 6 weeks before surgery and cell-reinfusion; she should have been considered for a further heart transplantation because of her poor prognosis. The 3^{rd} patient, although initially less severely affected, had progressively developed a deep congestive heart failure entailing life threatening at 3 months post-AMI.

TABLE 6
Patients' Data

| | | Nb of | | Time | | Nb of CD34+ |
|---|---|---|---|---|---|---|
| | | occluded | Infarct | between | Nb of | cells injected |
| Patients | Age/Sex | arteries | localisation | Infarct/TX | aphereses | (×10⁶) |
| 1 | 33 M | 3 | Antero-septal | 8 y | 2 | 29.1 |
| 2 | 49 F | 3 | Antero-apical | 6 w | 1 | 40.3 |
| 3 | 61 F | 1 | Antero-apical | 12 w | 2 | 43.8 |
| 4 | 33 M | 2 | Antero-apical | 6 w | 1 | 107.6 |
| 5 | 70 M | 2 | Antero-apical | 6 mo | 1 | 41.0 |
| | | (restenosis) | | | | |

Average 52.36

During a pre and a post-surgery period, all 3 patients well tolerated cell mobilization and collection procedures, without any side effect except transient mild thrombocytopenia. Adequate number of CD34⁺ cells was yielded with one apheresis in patient 2, when two were required in the others. Purity and viability of the CD34⁺ cell suspension were rather good in all cases. CABG was begun as soon as the cell graft was definitely available, and was done at beating heart. The cell suspension was infused through all the ischemic area by 8-10 longitudinal and parallel injections of 1.5-2 ml each, just before completion of the operation. Patients were immediately transferred to the intermediate care unit, and were finally discharged, as usual, to a rehabilitation program after 6-8 days. None has presented supraventricular arrhythmia up to 3 years after CABG. Patient 2 rapidly developed a relevant pericardial effusion, which is not rare after beating-heart surgery and was easily managed without any sequelae.

Post-surgery clinical evaluation was performed at 6 months and 3 years with the following results:

At 6 months:

TABLE 7
Posttransplant results

| | 6 months myocardial function Improvement | | | | | | | |
|---|---|---|---|---|---|---|---|---|
| | Petscan | | | | | | | |
| | | ILVEF | LVED (mm) | | Viability (nb | Perfusion (nb | | |
| | Early | before/6 | before/6 | | of segments | of segments | Area kinesis | NYHA grade |
| Patients | complications | months | months | Segment area | improved) | improved) | (− to +++) | before/6months |
| 1 | None | 34%/38% | 63/59 | Ant. Septal | 0/8 | 0/8 | − | IV/III |
| 2 | Pericardial | 30%/44% | 64/61 | Ant. | 6/8 | 5/8 | +++ | IV/I |
| | effusion | | | Apex | 1/1 | 1/1 | + | |
| 3 | None | 33%/53% | 47/43 | Ant. Lateral | 6/8 | 6/8 | ++ | IV/I |
| | | | | Apex | 0/1 | 0/1 | − | |
| 4 | None | 25%/NR | NR | NR | NR | NR | NR | IV/NR |
| 5 | None | 21%/NR | NR | NR | NR | NR | NR | IV/NR |

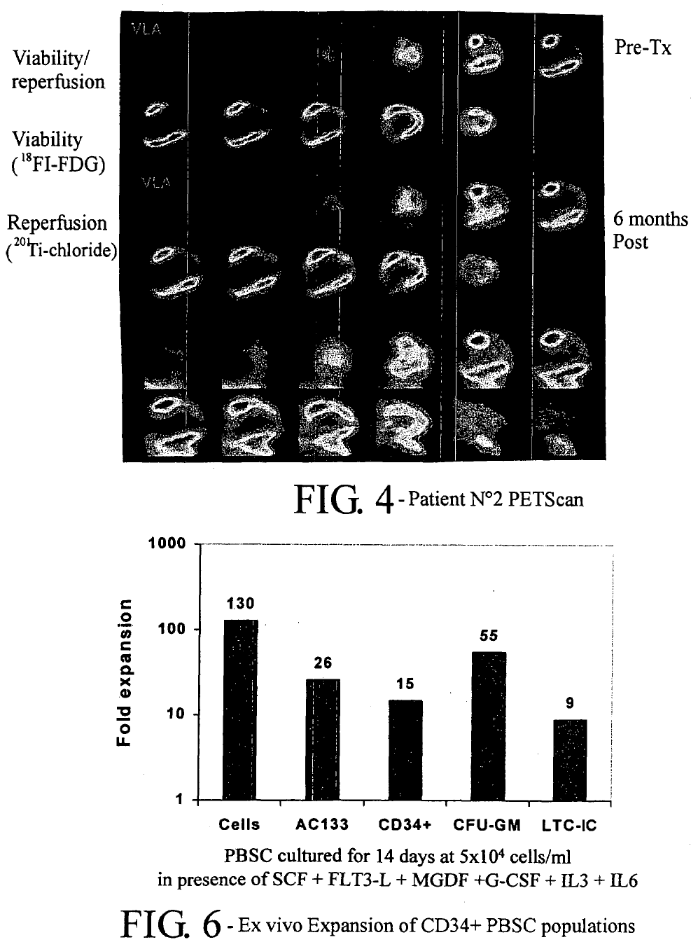

Patient 1 did not show any significant improvement of his cardiac function, even if his exercise capacity slightly increased, certainly only due to CABG. But we did not expect anything better indeed, as his 8-year-old infarcted zone appeared totally calcified at the time of cell reinfusion. Furthermore, he received the lowest amounts of CD34⁺ cells and subsets, compared to respectively 1.5 and 2-fold higher quantities of CD34⁺ cells and CD133⁺ subset reinfused in the other patients. Patient 2 cell-graft also contained the highest amounts of CD133⁺KDR⁺ and Desmin⁺ cells. Her 6-month-PETScan images showed striking improvements in viability and perfusion of the previously akinetic and non-surgically reperfused ischemic area (FIG. 4), correlated with major recovery of left anterior wall contractility, LVEF index and exercise capacity. Although at unequal degrees, patient 3 also showed an impressive improvement of most of these parameters.

At 3 years:

Patient 1 cardiac function has not significantly further improved: the left ventricular cavity appears even still more enlarged as compared with the onset (LVEDD=84 ml), LVEF remains between 36 and 46% depending on measurement incidences, in relation with a total akinesia of almost all the anterior wall, of the septo-apical area and of the apex. Only the lateral wall kinesis has improved, due to CABG.

On the contrary, cardiac function parameters of patients 2 and 3 still improved more:

In patient 2, LVEDD decreased at 55 ml, LVEF is now at 53% (+23% from the primitive base-line) whichever the measurement incidences. Contractility of the anterior wall and of the antero-septal junction is quite good, and even the apex kinesis is now significantly improved. The patient can now walk fast for at least 2 kms without dyspnea, and she works hard on a farm.

Patient 3 also shows a very significant improvement of the left ventricular function, with a LVEF at 64% (+31%, almost normal), a LVEDD of 31 ml, a normal kinesis of the median and the basal thirds of the antero-septal junction and of the septum, a mild hypokinesia of the anterior wall. Only the apex remains totally akinetic. She lives normally.

Thus, these preliminary data demonstrate that G-CSF administration, apheresis procedure, and intracardiac reinfusion of cell suspension volumes larger than those proposed by others are feasible and well tolerated in patients with a very severe AMI, which was confirmed in the new patients recently enrolled. “Purified” CD34 cells contain in various proportions, already dedifferentiated cells capable of facilitating either neoangiogenesis or striated muscle regeneration. Reinfusion of such cells in akinetic and not reperfused infarction area is followed by significant long-term improvements in its viability, perfusion and contractility. Whether it comes from these cells or not is not clear and it needs to be confirmed with more patients. However, such improvements are properly unusual after CABG without any surgical reperfusion, of the infarction area. Shortening the time between AMI and cell transplantation, and amounts of cell subsets reinfused are probably essential for potentially successful myocardium regeneration.

After this highly promising clinical phase-I trial a larger scale approach according to the present invention is undertaken.

To be able to answer on a large scale to the foreseeable increase in clinical practice of cellular cardiomyoplasty in a near future, several improvements of the current cell processing will be required. More particularly, leukapheresis procedure undoubtedly represents a restrictive factor in this way, at least for the following reasons:

a) It is likely that the sooner the cell be reinfused after AMI, the better the clinical results would be. Even if the protocol shows that apheresis procedure is clinically well tolerated when performed 6-12 weeks after AMI, it might not be the case in patients having undergone AMI only 8-10 days ago.

b) Performing apheresis sessions needs an authorized, well-equipped and well-trained team. Only a few medical centers presently answer such requirements, most being already overloaded with their current practice (HSC collection for hematological purpose). As each apheresis session needs approximately 3 hours, it will be difficult for them to assume much more sessions, and consequently to satisfactorily answer on a large scale to new demands.

c) Another restrictive factor is represented by coronary by-pass surgery. The aim is to reinfuse stem cells during such a surgery in a current Phase I study for ethical reasons only. But, of course, to propose cell cardiomyoplasty as a “routine” technology for heart therapy, as may be angioplasty and/or stenting, it is imperative to avoid CABG and use a less invasive way for cell reinfusion.

Thus, it is required to realize a new approach using a cell expansion process to yield enough CD34+ cells from relatively small total blood samples, avoiding then to perform leukapheresis.

Once defined that the final goal is still to improve the post-AM ischemic zone viability, reperfusion and contractility, and, consequently the patient's quality of life and survival, two main and several associated objectives have been determined.

a) Set up a cell expansion procedure allowing yielding as many cells as when achieved by leukapheresis, and thus avoid this relatively invasive procedure.

b) Maintain the cost of the complete cardiomyoplasty procedure at a minimal level representing the average cost for angioplasty and/or stenting.

And further,

a) Intend to treat around 5% of total severe cardiac failure over the 5-6 years to come.

b) Evaluate and justify savings potentially induced by cardiomyoplasty in terms of drugs, investigation, and lesser morbidity on a long-term follow-up.

c) Avoid coronary by-pass surgery, to be replaced by direct intra-ventricular cell reinjection.

The present invention attempts to offer a solution to simply, efficiently, reliably and at moderate cost, treatment of chronic heart failure by carrying out a cellular cardiomyoplasty process.

Therefore the method according to the present invention is based on the potential capacity of CD34+ cells to regenerate myocardium after AMI and on their collection in blood rather than in bone marrow after G-CSF mobilization, as in the above detailed current Phase I assay. Four major modifications differ from this assay:

a) G-CSF CD-34+ cell mobilization is started as soon as the infarct is stabilized and its impact on heart function has been evaluated. Clearly, G-CSF administration should begin 3-5 days after AMI.

b) Cells are collected on the 6^{th} day of G-CSF mobilization by withdrawing total blood at a final total volume of 200 ml by 3-4 sequential venous punctures within 12 hours.

c) Once withdrawn, blood samples are gathered and rapidly shipped to an agreed cell-processing laboratory for ex-vivo CD34+ cell selection and expansion within a two weeks-period to achieve around a 20-fold increase of the total number of CD34⁺ cells.

d) Resuspension of the amplified cell product in a final volume of 10 ml of autologous plasma, and packaging of the cell suspension in a sterile syringue which will be shipped back to the cardiology center in charge of the patient, and reinjected within 12 hours following packaging.

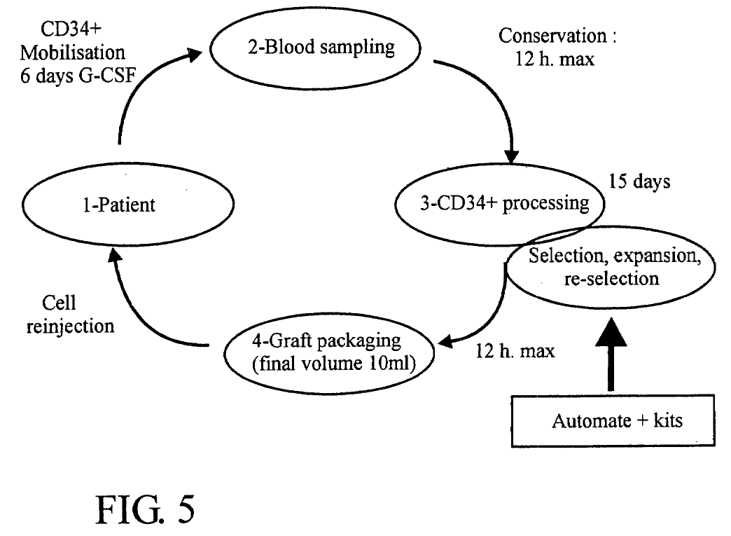

The full concept is summarized in FIG. 5. The following steps are represented:

1.—The process begins with GCSF-mobilization of CD34⁺ cells for 5 days.

2.—Blood sampling step (200 ml total volume) on the 6^{th} day of mobilization and shipping to the processing laboratory.

3.—CD34+ processing during 15 days: primary selection, expansion and secondary selection using an automated bio-reactor device.

4.—Graft packaging (10 ml volume) and shipping to the cardiology center.

5.—Cells reinjection to the patient.

This renewed approach would provide major advantages as compared with our current assay; these advantages include:

→Blood withdrawing is not painful and not stressful for the patient. Overall, it avoids performing leukapheresis procedure that could have unpleasant side effects when performed soon after AMI. Furthermore, it can be easily performed anywhere by a nurse.

→Blood withdrawing cost is very low in comparison with that of a leukapheresis procedure and would balance with the over-cost induced on the other side by cell expansion processing.

→The schedule of the total process, from G-CSF administration to the final cell product, would allow reinfusing the cells in the myocardium between the 24^{th} and the 26^{th} day (FIG. 5) after AMI. At this point, post-infarct ischemic tissues are still inflammatory which would favor intra-myocardium cell diffusion. On the contrary, once scar is definitely constituted, fibrosis tissue texture would prevent cell diffusion and intra-myocardial homing (see data for the 1^{st} patient enrolled in our current study). Thus the efficiency of the reinfused cells in repairing myocardium should be amplified by the precocity of the procedure.

A very efficient methodology for ex vivo CD34⁺ cell expansion with cells yielded either from BM, PBSC, or CB was finalized a few years ago. It is likely the only method published so far allowing the expansion of very immature stem cells in significant proportions. This method has been published in 2000 (Kobari L. et al. In vitro and in vivo evidence for the long-term multilineage myeloid, B, NK and T) reconstitution capacity of ex vivo expanded human CD34+ cord blood cells. Exp Hematol 2000; 28:1470-1480). A worldwide patent protects this expansion method (N° FR00/01311—May 16, 2000).

Thus the method for clinical use according to the invention is defined hereunder:

Briefly, once purified by immuno-selection, CD34⁺ cells are suspended at 10⁴ cells/ml in serum free long-term culture medium (LTCM) supplemented with Flt3-ligand (FL, 100 ng/ml, Valbiotech) and Stem Cell Factor (SCF, 100 ng/ml), Megakaryocyte Growth and Development Factor (MGDF, 100 ng/ml) and G-CSF (10 ng/ml), IL6 and IL3. The cell suspensions are incubated at 37° C. in a 5% C0₂/95% air atmosphere for 14 days, after which the cells are collected, washed in Iscove-Modified Dulbecco's Medium (IMDM, Seromed, Biochrom), counted by trypan blue exclusion and analyzed for progenitor/stem cells, immunophenotype and NOD-SCID engraftment.

Culture assays are performed in gas-permeable polypropylene bags (11.2 cm×7.5 cm, PL2417) kindly provided by J. Bender (Nexel, USA). Selected cells are seeded in 4 ml of complete LTCM according to the manufacturer's recommendations and 16 ml of fresh medium containing cytokines are added to each bag on day 6. According to conditions determined in previous studies, the cells are removed on day 14 with a syringe and washed in IMDM prior to analysis.

Cell expansion is expressed as the fold increase, which is calculated by dividing the output absolute number of cells, progenitors and LTC-IC after 14 days expansion by the respective input number on day 0.

High level of expansion of total cells and of progenitor cells are obtained with this method, with median ranges of 130-fold for total cells, 15-20 fold for CD34⁺ cells, 26-fold for AC133⁺ cells and almost 10-fold for LTC-IC respectively (FIG. 6). Moreover, the qualities of the CFC and LTC-IC progenitors in expanded and non-expanded cells are similar. Also, the telomere length, which is considered to be a marker of the cellular proliferation potential, was unchanged in CD34⁺ cells despite a mean 15-fold expansion (see FIG. 6).

Moreover, expanded CD34⁺ cells retain their ability to engraft sub-lethally irradiated NOD-SCID mice, together with their capacity to support long-term hematopoiesis and multipotent differentiation into myeloid and B-, NK- and T-lymphoid cells.

All these data constitute a strong rational for the clinical use of ex vivo expanded CD34⁺ cells.

Number of Cells Expected in the Final Graft Product:
on average, 30 cells/μl can be reasonably expected in total blood after G-CSF mobilization. For a total blood withdrawing of 200 ml, it would represent a total yield of 6×10⁶ CD34⁺ cells on average.
cell expansion procedure would achieve a 15-20 fold increase of CD34+ cells, which would allow obtaining a total of CD34⁺ cells ranging between 9×10⁷ and 1.2×10⁸.
2 immuno-selection steps are required during the procedure: the first one to purify CD34⁺ cells from total blood before expansion procedure, the second occurring after expansion procedure to select expanded CD34⁺ cells among more mature expanded cells. Both together, these 2 immuno-selection procedures would entail a loss of about 30% CD34⁺ cells. Thus, the final number of CD34⁺ cells contained in the graft product to be administered to the patient would approximately range between 6 and 8×10⁷ cells. If we consider that in our current protocol we reinfuse in our patients around 4×10⁷ CD34⁺ cells as an average, this expected amount should be enough to ensure a potential myocardial regeneration.

1. Cellular cardiomyoplasty process based on the potential capacity of CD34⁺ cells to regenerate myocardium after acute myocardial infarct (AMI) and on their collection in blood in which the following phases are performed:
Phase 1 a G-CSF-mobilization phase of CD-34+ cell is started as soon as the infarct is stabilized and its impact on heart function has been evaluated;
Phase 2 a cells collecting phase is undertaken after G-CSF-mobilization;
Phase 3 a cells processing phase is performed to select ex-vivo CD34+ cells and expand them in vitro to achieve around a 20-fold increase of the total number of CD34⁺ cells;
Phase 4 a resuspension phase of the amplified-cell product in a final predetermined volume of autologous plasma; and
Phase 5 a packaging phase of the cell suspension in a sterile syringue for reinjection to the patient.

2. Process according to claim 1, in which the G-CSF administration is started at least between 3-5 days after AMI.

3. Process according to claim 1, in which the cells collection phase is undertaken at least on the 6^{th} day of G-CSF mobilization.

4. Process according to claim 1, in which the cells collection phase is performed by withdrawing total blood at a final total volume of 200 ml.

5. Process according to claim 1, in which the cell collection phase is performed by several sequential venous punctures within 12 hours.

6. Process according to claim 4, in which the cell collection phase is performed by at least 3 to 4 sequential venous punctures.

7. Process according to claim 1, in which the in vivo expansion of the CD 34+ cells of phase 3 is performed during a two weeks period.

8. Process according to claim 1, in which the resuspension phase is performed in a final predetermined volume of between 5 and 15 ml and preferably 10 ml of autologous plasma.

9. Process according to claim 1, in which the reinjection of phase 5 is performed within between 5 and 18 and preferably about 12 hours following packaging.
